(19) United States
(12) Patent Application Publication
(10) Pub. No.: US 2021/0046123 A1
(43) Pub. Date: Feb. 18, 2021

(54) METHODS FOR TREATING DISEASE ASSOCIATED WITH SENESCENCE

(71) Applicant: Steadman Philippon Research Institute, Vail, CO (US)

(72) Inventors: Johnny HUARD, Edwards, CO (US); Marc Joseph PHILIPPON, Edwards, CO (US); Sudheer Kumar RAVURI, Avon, CO (US); William Sealy HAMBRIGHT, Vail, CO (US); Charles HUARD, Edwards, CO (US); Michael Terrance MULLEN, II, Eagle-Vail, CO (US); John Weston MITCHELL, Eagle-Vail, CO (US); Kaitlyn Elizabeth WHITNEY, Eagle-Vail, CO (US)

(21) Appl. No.: 16/994,356

(22) Filed: Aug. 14, 2020

Related U.S. Application Data
(60) Provisional application No. 62/887,090, filed on Aug. 15, 2019.
(60) Provisional application No. 62/890,893, filed on Aug. 23, 2019.
(60) Provisional application No. 62/890,910, filed on Aug. 23, 2019.
(60) Provisional application No. 62/959,012, filed on Jan. 9, 2020.
(60) Provisional application No. 62/985,242, filed on Mar. 4, 2020.

(51) Int. Cl. A61K 35/28; A61K 31/366; A61P 43/00; A61P 19/02; G01N 33/50; G01N 21/64

(52) U.S. Cl. CPC A61K 35/28; A61K 31/366; A61P 43/00; A61P 19/02; G01N 2021/6439; G01N 21/6428; G01N 2333/938; G01N 2458/30; G01N 33/5091

(57) ABSTRACT

The present disclosure provides method of detecting senescent cells in a cell sample and methods of treating a disease, disorder, or condition associated with senescence in a subject by administering at least one senolytic agent to the subject.

This application is being filed on Aug. 14, 2020 as a U.S. Nonprovisional Patent Application and claims the benefit of priority to U.S. Provisional Patent Application Nos. 62/887,090, filed Aug. 15, 2019; 62/890,893, filed Aug. 23, 2019; U.S. Pat. No. 62,890,910, filed Aug. 23, 2019; 62/959,012, filed Jan. 9, 2020; and 62/985,242, filed Mar. 4, 2020, the disclosures of each herein incorporated by reference in their entireties.

### FIELD OF THE DISCLOSURE

The present disclosure relates to the use of senolytic agents to clear senescent cells. The disclosure further relates to methods for treating diseases and disorders associated with senescence.

### BACKGROUND

In cellular senescence, normally proliferating cells are in a permanent state of cell cycle arrest, in which they no longer respond to growth stimuli and no longer divide. Cell cycle arrest in progenitor cells contributes to a loss in the capacity to repair tissue. Furthermore, senescent cells produce pro-inflammatory and matrix-degrading molecules, a response referred to as the senescence-associated secretory phenotype (SASP) (Childs, et al. 2015 Nat Med 21(12):1424-1435; Paez-Ribes, et al. 2019 EMBO Mol Med 11:1-19). Cellular senescence has been associated with the aging process and with age-related disease. As a result, targeting senescent cells has emerged as a therapeutic approach.

### BRIEF SUMMARY OF THE INVENTION

In one aspect, the invention provides a method of detecting senescent cells in a cell sample, the method comprising staining the cells with C12FDG; and subjecting the cells to flow cytometry. In one embodiment, the cell sample comprises CD3⁺ T cells. In another embodiment, the cell sample is total peripheral blood mononuclear cells, bone marrow aspirate (BMA), whole blood, ex vivo culture-expanded stem cells, banked stem cells, freshly isolated stem cells, or endogenous stem cells. In still another embodiment, the detected senescent cells are characterized according to stage of senescence. In still another embodiment, the characterization is based on brightness of signal. In a further embodiment, the stage of senescence is early-stage (relatively low C12FDG positivity, “dim”, low green fluorescent intensity on a flow cytometry plot), mid-stage (relatively moderate C12FDG positivity), or late-stage (relatively high C12FDG positivity, “bright”, high fluorescent intensity on a flow cytometry plot), as determined by normalized event gating with flow cytometry. In certain embodiments, the late-stage senescent cells are the target of the senolytic agent or the senotherapeutic.

In another aspect, the invention provides a method of removing senescent cells from banked stem cells, the method comprising adding at least one senolytic agent to the cells.

In still another aspect, the invention provides a method of enriching banked stem cells, the method comprising adding at least one senolytic agent to the cells.

In certain embodiments of methods according to the invention, the banked stem cells are selected from the group consisting of ADSCs, ex vivo culture-expanded stem cells, freshly isolated stem cells, endogenous stem cells, bone marrow aspirate concentrate (BMAC) stem cells, and whole blood stem cells. In additional embodiments, at least 25% of the senescent cells are removed, at least 50% of the senescent cells are removed, at least 75% of the senescent cells are removed, at least 80% of the senescent cells are removed, at least 85% of the senescent cells are removed, at least 90% of the senescent cells are removed, or at least 95% of the senescent cells are removed.

In yet another aspect, the invention provides a method of treating a disease, disorder, or condition associated with senescence in a subject, the method comprising administering at least one senolytic agent to the subject.

In a further aspect, the invention provides a method of treating a disease, disorder, or condition associated with senescence in a subject, the method comprising removing stem cells from the subject, culturing the cells with at least one senolytic agent, and administering the cultured cells to the subject.

In still a further aspect, the invention provides a method of treating an age-related disease, disorder, or condition in a subject, the method comprising removing stem cells from the subject, culturing the cells with at least one senolytic agent, and administering the cultured cells to the subject.

In certain embodiments of methods according to the invention, the disease, disorder, or condition is osteoarthritis.

In another aspect, the invention provides a method of improving a surgical outcome in a subject, the method comprising administering at least one senolytic agent to the subject. In one embodiment, the senolytic agent is administered to the surgical site in the subject.

In still another aspect, the invention provides a method of improving a surgical outcome in a subject, the method comprising removing stem cells from the subject, culturing the cells with at least one senolytic agent, and administering the cultured cells to the subject. In one embodiment, the cultured cells are administered to the surgical site in the patient.

In certain embodiments of methods according to the invention, the surgical outcome is wound healing. In additional embodiments of methods according to the invention, the surgical site is a wound.

In another aspect, the invention provides a method of removing senescent cells from an orthobiologic product, the method comprising adding at least one senolytic agent to the product. The orthobiologic product may be selected, without limitation, from the group consisting of a bone graft, autologous blood, platelet-rich plasma (PRP), autologous conditioned serum, stem cells, Bone Marrow Aspirate Concentrate (BMAC), Platelet Rich Plasma (PRP) PRP, Alpha 2 Macroglobulin (A2M), amniotic fluid, placental tissue, umbilical cord tissue, and hyaluronic acid.

In certain embodiments of methods according to the invention, the at least one senolytic agent is Fisetin.

In one aspect, the invention provides a pharmaceutical composition comprising at least one senolytic agent for use in the treatment of an age-related disease, disorder, or condition.

In another aspect, the invention provides a pharmaceutical composition comprising at least one senolytic agent for use in the treatment of a disease, disorder, or condition associated with senescence.

In still another aspect, the invention provides a pharmaceutical composition comprising at least one senolytic agent for use in the improvement of a surgical outcome.

In certain embodiments of compositions according to the invention, the at least one senolytic agent is Fisetin.

In one aspect, the invention provides a method of treating a subject having a senescence-related condition or disease, the method comprising measuring senescence in the subject or in a tissue sample from the subject and administering a senolytic agent to the subject. In one embodiment, the subject or the tissue sample of the subject comprises at least about 4% senescent cells. In another embodiment, the subject or the tissue sample of the subject comprises at least about 4% to about 8% senescent cells. In another embodiment, the senolytic agent is Fisetin.

In embodiments of the disclosure that specify the selection of “at least one . . . selected from the group consisting of” or simply “selected from the group consisting of”, the use of the conjunction “and” between the final two items of the list following such language indicates that the items in the sequence are alternatives to one another, and that one (or more) of these items is/are selected. It does not mean that each of the items is necessarily selected.

Other embodiments of the present invention will become apparent from a review of the ensuing detailed description.

### BRIEF DESCRIPTION OF THE FIGURES

FIGS. 1A-1E show the effect of serial passaging on cellular senescence in ADSCs. FIG. 1A graphically depicts the percent of cells stained with C12FDG at each passage number (p3, p4, p6, and p8). FIGS. 1B-1E are representative histogram plots comparing unstained controls to C12FDG stained ADSCs at passage 3 (FIG. 1B), passage 4 (FIG. 1C), passage 6 (FIG. 1D), and passage 8 (FIG. 1E). R1 regions indicate cells positive for C12FDG. One-way ANOVA results show all consecutive passages are significantly different (p<0.0001).

FIGS. 2A and 2B show the effect of serial passaging on cellular senescence in ADSCs. The bar graph in FIG. 2A depicts the senescence associated secretory phenotype (SASP)—factors IL-6, IL-8, and MCP-1—increasing in concentration with passage number (p6 to p8). FIG. 2B provides a plot of the % of cells positive for senescence-associated heterochromatin foci (SAHF H3K9 and H2AX) increasing with passage number (p4 to p18). Two-way ANOVA shows SASPs increase significantly (**=p<0.01, ****=p<0.0001). One-way ANOVA shows SAHFs increase significantly (**=p<0.01).

FIG. 3 shows, in bar graph form, the percentage of viable cells at varying Fisetin concentrations (50 μM, 25 μM, and 1 μM) for young female (YF), old female (OF), young male (YM), and old male (OM) subjects. Values are relative to untreated, control cells. One-way ANOVA shows no significant difference in the percentage of viable cells among treatments.

FIGS. 4A-4D show that Fisetin treatment reduces β-galactosidase via C12FDG staining. The plot of FIG. 4A depicts the % cells positive for C12FDG at passages 6, 8, and 10 with or without Fisetin treatment. The percent of cells stained positive for C12FDG with and without treatment was reduced at every passage. FIGS. 4B-4D show representative histogram plots comparing untreated controls to Fisetin-treated ADSCs at passage 6 (FIG. 4B), passage 8 (FIG. 4C), and passage 10 (FIG. 4D). R1 regions indicate cells positive for C12FDG. One-way ANOVA statistics show significant differences with each treatment (*=p<0.05, **=p<0.01, ***=p<0.001)

FIGS. 5A-5C show that Fisetin treatment reduces H3K9 and [symbol] -H2AX Senescence Associated Heterochromatin Foci (SAHF). The percent reduction of cells staining positive for H3K9 and [symbol] -H2AX for OF (old female) vs. YF (young female) treated with Fisetin or untreated at passages 4 and 18 is summarized in FIG. 5A. The results are provided in the plots of FIG. 5B (for passage 4) and FIG. 5C (for passage 18), as well. One-way ANOVA shows significant reduction in SAHFs (*=p<0.05, **=p<0.01, ****=p<0.0001).

FIG. 6 shows a bar graph depicting the concentrations of SASP factors IL-6, IL-8, and MCP-1 for untreated cells vs. cells treated with Fisetin. One-way ANOVA statistics show significant differences with each treatment (*=p<0.05, **=p<0.01)

FIG. 7 schematically depicts the protocol for T-cell enrichment using Stem Cell Technologies Sepmate™ tubes and RosetteSep™ Chemistry.

FIG. 8 shows an example of the results of the detection of senescent cells using C12FDG staining and flow cytometry. The entire event cells resulted from optimized setting parameters and were taken into account for further evaluation of the subsets of cells. Background cells were excluded/dumped. Subsets were categorized as early stage senescent cells, mid-stage senescent cells, and late stage senescent cells.

FIGS. 9A, 9B, and 9C are flow cytometry plots (dot/density plots and a histogram plot) depicting populations of senescent cells. In FIG. 9A, it is clarified that cell size increases with the y axis, and that intensity of senescence increases with the x axis.

FIG. 9B shows cell populations corresponding to mid-stage senescence (“R2”) and late-stage senescence (“R1”). The histogram plot of FIG. 9C presents that data of FIG. 9B in another way, further quantitating the percentage of mid-stage (24.6%) vs. late-stage (3.5%) senescent cells.

FIGS. 10A and 10B show flow cytometry dot/density plots. FIG. 10A shows the plots for a 25-year-old male, a 55-year-old male, and an 85-year-old male. FIG. 10B shows the plots for 41-year-old female and a 77-year-old female.

FIG. 11A shows a sample senescence profiling result for a subject. The flow cytometry results are provided as a dot plot, raw data, and cell count averages. The dim and bright senescent cells are visualized and quantified. FIG. 11B depicts, in bar graph form, the amount of dim vs. bright senescent cells across a subset of study subjects.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present disclosure, the preferred methods and materials are now described. All publications mentioned herein are incorporated herein by reference to describe in their entirety.

### Senescent Cells

Senescent cells include, without limitation, senescent preadipocytes, senescent endothelial cells, senescent fibroblasts, senescent neurons, senescent epithelial cells, senescent chondrocytes, senescent mesenchymal cells, senescent macrophages, and senescent smooth muscle cells.

Senescent cells and senescent cell-associated molecules can be detected by techniques and procedures described in the art. For example, the presence of senescent cells in tissues can be analyzed by histochemistry or immunohistochemistry techniques that detect the senescence marker, SA-β galactosidase (SA-β gal) (Dimri, et al. 1995 Proc. Natl Acad. Sci. USA 92:9363-9367; Lee, et al. 2006 Aging Cell 5(2):187-195). The presence of the senescent cell-associated polypeptide p16, specifically, p16INK4a and p21Cip1, can be determined by immunochemistry methods practiced in the art, such as immunoblotting analysis (Dimri, et al. 1996 Biol. Signals 5:154-162). Expression of p16 mRNA in a cell can be measured by techniques practiced in the art including quantitative PCR. The presence and level of senescence cell associated polypeptides (e.g., polypeptides of the SASP, generally called SASP factors or proteins, or senescence messaging secretome (SMS) can be determined by using automated and high throughput assays.

The presence of senescent cells can also be determined via detection of senescent cell-associated molecules, which include growth factors, proteases, cytokines (e.g., inflammatory cytokines), chemokines, cell-related metabolites, reactive oxygen species (e.g., H₂O₂), and other molecules that stimulate inflammation and/or other biological effects or reactions that may promote or exacerbate the underlying disease of the subject.

### Senolytic Agents

Senolytic agents are agents that selectively target and induce apoptosis/death of senescent cells (Kirkland, et al. 2017 J Am Geriatr Soc 65(10):2297-2301; Zhu, et al. 2015 Aging Cell 14(4):644-658). Senolytic agents include, without limitation, flavonoids (quercetin, Fisetin), tyrosine kinase inhibitor (e.g., dasatinib)+quercetin, alkanoids (piperlongumine), curcumin analog, navitoclax, 17-DMAG, BCL-2-targeting agents (ABT-263, ABT-737), and combinations thereof. Specifically, these agents target senescent cell anti-apoptotic pathways (SCAPs), which are upregulated during senescence. Senolytic agents are sometimes included in a group of interventions known as “geroprotectors” or “senotherapies”. Senolytic agents also include geroprotective nutrients such as, without limitation, myricetin, N-acetyl-cysteine (NAC), gamma tocotrienol, or epigallocatechin-gallate (EGCG).

### Senescence Markers/SASP

Senescent cell markers include, without limitation, increased cell size, accumulation of lipofuscin, high expression of cell cycle regulators (e.g., p16INK4A), p21CIP1, and senescence associated secretory phenotype (SASP) factors (including, without limitation, TNF-alpha, interleukin-6 (IL-6), multifunctional cytokine IL-1beta, chemokines CXCL10, RANTES/CCL5, and MCP-1, matrix metalloprotease MMP3, and serine-protease inhibitor PAI-1 (Senescence Associated Secretory Phenotype (SASP): TNF-alpha, interleukin-6 (IL-6), the multifunctional cytokine IL-1β, the chemokines CXCL10, RANTES/CCL5 and MCP-1, the matrix metalloprotease MMP3, and the serine-protease inhibitor PAI-1 (Tchkonia, et al. 2013 J Clin Invest 123, 966-72; Sun, et al. 2018 Trends Mol Med 24, 871-885; Rao and Jackson 2016 Trends Cancer 2:676-687)), increased cellular senescence-associated β-galactosidase (SA-βgal) activity/accumulation of β-galactosidase, hemostatic factors (e.g., PAI-1), proteases, formation of senescence-associated heterochromatin foci (SAHF), and the appearance of senescent-associated distension of satellites (SADS) and telomere-associated DNA damage foci (TAFs) (Kirkland, et al. 2017 J Am Geriatr Soc 2017 65(10):2297-2301; Coppe, et al. 2010 ARP 5:99-118; Young and Narita 2009 EMBO Rep 10:228-30).

Senescence-associated secretory phenotype (SASP) refers to a phenotype that often develops in senescent cells, marking the dramatic changes in their secretome (Coppe, et al. 2010 ARP 5:99-118; Young and Narita 2009 EMBO Rep 10:228-30). It entails the release of pro-inflammatory cytokines, bradykines, and chemokines, prostanoids, miRNAs, damage-associated molecular pattern proteins (DAMPs), tissue-damaging proteases (i.e., metalloproteases (MMPs)), factors that impact stem and progenitor cell function, hemostatic factors, and growth factors (Kirkland, et al. 2017 J Am Geriatr Soc 2017 65(10):2297-2301). SASP factors include, without limitation, interleukins (IL-6, IL-8, IL-1β), monocyte chemoattractant protein-1, and plasminogen-activated inhibitor-1.

Senescent cell-associated molecules include those that are described in the art as comprising the senescence-associated secretory phenotype, senescent-messaging secretome, and DNA damage secretory program (DDSP). These groupings of senescent cell associated molecules, as described in the art, contain molecules in common and are not intended to describe three separate distinct groupings of molecules. Senescent cell-associated molecules include certain expressed and secreted growth factors, proteases, cytokines, and other factors that may have potent autocrine and paracrine activities (see, e.g., Coppe, et al. 2006 J. Biol. Chem. 281:29568-74; Coppe, et al. 2010 PLOS One 5:99-118; Krtolica, et al. 2001 PNAS U.S.A. 98:12072-77; Parrinello, et al. 2005 J. Cell Sci. 118:485-96). Extracellular matrix (ECM)-associated factors include inflammatory proteins and mediators of ECM remodeling that are strongly induced in senescent cells (see, e.g., Kuilman, et al. 2009 Nature Reviews 9:81-94). Other senescent cell-associated molecules include extracellular polypeptides (proteins) described collectively as the DNA damage secretory program (DDSP) (see, e.g., Sun, et al. 2012 Nature 18:1359-1368). Senescent cell-associated proteins also include cell surface proteins (or receptors) that are expressed on senescent cells, which include proteins that are present at a detectably lower amount or are not present on the cell surface of a non-senescent cell. Senescent cell-associated proteins also include cell surface markers, like cluster differentiation markers, that have modified expression (loss or gain) during senescence progression of certain cell types (ex. CD26, CD28).

Senescence cell-associated molecules include secreted factors that may make up the pro-inflammatory phenotype of a senescent cell (e.g., SASP). These factors include, without limitation, GM-CSF, GROα, GROαβγ, IGFBP-7, IL-1γ, IL-6, IL-7, IL-8, MCP-1, MCP-2, MIP-1α, MMP-1, MMP-10, MMP-3, Amphiregulin, ENA-78, Eotaxin-3, GCP-2, GITR, HGF, ICAM-1, IGFBP-2, IGFBP-4, IGFBP-5, IGFBP-6, IL-13, IL-10, MCP-4, MIF, MIP-3a, MMP-12, MMP-13, MMP-14, NAP2, Oncostatin M, osteoprotegerin, PIGF, RANTES, sgp130, TIMP-2, TRAIL-R3, Acrp30, angiogenin, Axl, bFGF, BLC, BTC, CTACK, EGF-R, Fas, FGF-7, G-CSF, GDNF, HCC-4, I-309, IFN-γ, IGFBP-1, IGFBP-3, IL-1 R1, IL-11, IL-15, IL-2R-α, IL-6R, 1-TAC, Leptin, LIF, MMP-2, MSP-a, PAI-1, PAI-2, PDGF-BB, SCF, SDF-1, sTNF RI, sTNF RII, Thrombopoietin, TIMP-1, tPA, uPA, uPAR, VEGF, MCP-3, IGF-1, TGF-β3, MIP-1-δ, IL-4, FGF-7, PDGF-BB, IL-16, BMP-4, MDC, MCP-4, IL-10, TIMP-1, Fit-3 Ligand, ICAM-1, Axl, CNTF, INFγ, EGF, BMP-6. Additional identified factors, which include those sometimes referred to in the art as senescence messaging secretome (SMS) factors, some of which are included in the listing of SASP polypeptides, include, without limitation, IGF1, IGF2, and IGF2R, IGFBP3, IDFBP5, IGFBP7, PA11, TGF-β, WNT2, IL-1a, IL-6, IL-8, and CXCR2-binding chemokines. Cell-associated molecules also include, without limitation, the factors described in Sun, et al., Nature Medicine, and include, for example, products of the genes, MMP1, WNT16B, SFRP2, MMP12, SPINK1, MMP10, ENPP5, EREG, BMP6, ANGPTL4, CSGALNACT, CCL26, AREG, ANGPT1, CCK, THBD, CXCL14, NOV, GAL, NPPC, FAM150B, CST1, GDNF, MUCL1, NPTX2, TMEM155, EDN1, PSG9, ADAMTS3, CD24, PPBP, CXCL3, MMP3, CST2, PSG8, PCOLCE2, PSG7, TNFSF15, C17orf67, CALCA, FGFJ8, IL8, BMP2, MATN3, TFP1, SERPINI 1, TNFRSF25, and IL23A (Coppe, et al. 2010 ARP 5:99-118; Young and Narita 2009 EMBO Rep 10:228-30; Basisty, et al. 2020 PLOS Biologyl 8(1):e300599). Senescent cell-associated proteins also include cell surface proteins (or receptors) that are expressed on senescent cells, which include proteins that are present at a detectably lower amount or are not present on the cell surface of a non-senescent cell.

### SASP Inhibitors

SASP inhibitors are agents that neutralize the senescence-associated secretory phenotype (SASP). SASP inhibitors are sometimes referred to as senostatic agents, which agents modulate a proinflammatory secretome and supplement senolytics for targeting aging and age-related diseases. Senostatic agents selectively suppress the deleterious effects of senescence (Kang 2019 Mol Cells 42(12):821-827). SASP inhibitors include, without limitation, metformin, rapamycin, JAK1/2 inhibitors (e.g., ruxolitinib), and glucocorticoids. In certain embodiments, one or more SASP inhibitors can be used in combination with one or more senolytic agents.

### Methods for Treating or Ameliorating Age-Related Diseases and Conditions

The present disclosure includes methods for treating, ameliorating, or preventing (i.e., reducing the likelihood of occurrence of) age-related diseases and conditions. Age-related diseases and conditions include conditions associated with senescence (Kirkland, et al. 2017 J Am Geriatr Soc 65(10):2297-2301; Zhu, et al. 2015 Aging Cell 14(4):644-658). The present disclosure also includes methods for treating, ameliorating, or preventing (i.e., reducing the likelihood of occurrence of) at least one symptom or indication an age-related disease or disorder in a subject. The methods according to this aspect of the invention comprise administering a pharmaceutical composition comprising a therapeutically effective amount of a senolytic agent to the subject in need thereof. In further embodiments, the composition additionally comprises at least one senostatic agent. The age-related diseases and conditions are, in further embodiments, conditions, diseases, or disorders related to, associated with, or caused by cellular senescence. Senescent cells can be the nexus of multiple disease conditions (Bhatia-Dey, et al. 2016 Front Genet 7:13).

As used herein, the terms “treat”, “treating”, or the like, mean to alleviate symptoms, eliminate the causation of symptoms either on a temporary or permanent basis, or to prevent or slow the appearance of symptoms of an age-related disease or condition in a subject. In certain embodiments, the age-related diseases or conditions include, without limitation, cancer, tumorigenesis, metastasis, fibrosis, cardiovascular disease, myocardial infarction, atherosclerosis, cardiomyocyte hypertrophy, heart failure, peripheral vascular disease, premature aging of the hematopoietic system, obesity, obesity-induced metabolic syndrome, adipose atrophy, type 1 diabetes, type 2 diabetes, sarcopenia, inflammatory disease, osteoarthritis, degenerative joint disease, kyphosis, osteoporosis, loss of bone mass, herniated intervertebral discs, rheumatoid arthritis, irritable bowel syndrome, inflammatory bowel disease, glaucoma, cataracts, pulmonary disease, pulmonary insufficiency, idiopathic pulmonary fibrosis (IPF), sarcopenia, kidney dysfunction/renal failure, tau-dependent pathologies, neurological disorders, neurodegeneration, motor function diseases, cerebrovascular disease, emphysema, inflammatory disorders of the skin, and natural aging. In additional embodiments, the age-related disease or condition is slow healing, including after injury (wound healing) or surgery (post-procedure healing, response to physical therapy).

As used herein, the expression “a subject in need thereof” means a human or non-human mammal that exhibits one or more symptoms or indications of an age-related disease or condition, and/or who has been diagnosed with an age-related disease or condition. Throughout the present disclosure, the terms “subject”, “patient”, and “subject in need thereof” are used interchangeably. The term “a subject in need thereof” may also include, e.g., patients who, prior to treatment, exhibit (or have exhibited) one or more indications of an age-related disease or condition, and/or who exhibit senescence biomarkers and/or SASP. The term “a subject in need thereof” may also include a patient who is going to undergo treatment and/or surgery, for example, orthopedic surgery.

### Pharmaceutical Compositions

The present invention includes methods that comprise administering at least one senolytic agent to a subject, wherein the agent is contained within a pharmaceutical composition. The pharmaceutical compositions of the invention may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., a bioengineered scaffold, encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intra-articular, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In a preferred embodiment, the composition is administered orally.

In certain situations, the pharmaceutical composition can be delivered in a controlled release system. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

Injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the agent in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc.

Pharmaceutical compositions for oral or parenteral use are prepared into dosage forms in a unit dose suited to fit a dose of the active ingredients. Such dosage forms in a unit dose include, for example, tablets, pills, capsules, injections (ampoules), suppositories, etc.

### Administration Regimens

The present invention includes methods comprising administering to a subject at least one senolytic agent at a dosing frequency of at least once. In additional embodiments, the inventive methods comprise administering to a subject at least one senolytic agent at a dosing frequency of more than once. In still further embodiments, dosing is such that a therapeutic response is achieved. The therapeutic response in this context constitutes a reduction in senescent cells. In specific embodiments, dosing of the senolytic agent is chronic or acute, but still transient in nature (for example, not taken daily) to minimize potential side effects from daily chronic treatment. In one embodiment, a senolytic drug is administered orally twice per month for one month (acute). In a further embodiment, Fisetin is administered orally 2 daily doses back-to-back, followed by 28 days off routinely for years or decades with a transient regimen (chronic).

According to certain embodiments of the present invention, multiple doses of at least one senolytic agent may be administered to a subject over a defined time course. The methods according to this aspect of the invention comprise sequentially administering to a subject multiple doses of the agent. As used herein, “sequentially administering” means that each dose of the agent is administered to the subject at a different point in time, e.g., on different days separated by a predetermined interval (e.g., hours, days, weeks or months). The present invention includes methods that comprise sequentially administering to the patient a single initial dose of the agent, followed by one or more secondary doses of the agent, and optionally followed by one or more tertiary doses of the agent.

The terms “initial dose,” “secondary doses,” and “tertiary doses,” refer to the temporal sequence of administration of the at least one senolytic agent. Thus, the “initial dose” is the dose that is administered at the beginning of the treatment regimen (also referred to as the “baseline dose”); the “secondary doses” are the doses that are administered after the initial dose; and the “tertiary doses” are the doses that are administered after the secondary doses. The initial, secondary, and tertiary doses may all contain the same amount of agent, but generally may differ from one another in terms of frequency of administration. In certain embodiments, however, the amount of agent contained in the initial, secondary and/or tertiary doses varies from one another (e.g., adjusted up or down as appropriate) during the course of treatment.

The methods of the present invention, according to certain embodiments, comprise administering to the subject a second therapeutic agent in combination with the at least one senolytic agent. As used herein, the expression “in combination with” means that the second therapeutic agent is administered before, after, or concurrent with the senolytic agent. The term “in combination with” also includes sequential or concomitant administration of the senolytic agent and the additional therapeutic agent.

### Dosage

The amount of senolytic agent administered to a subject according to the methods of the present invention is, generally, a therapeutically effective amount. As used herein, the phrase “therapeutically effective amount” means an amount of senolytic agent that results in one or more of: (a) a measurable reduction in senescent cells; and (b) an improvement in a symptom of an age-related disease or condition. In one embodiment, the condition is the healing of a wound. In another embodiment, the condition is osteoarthritis or related orthopaedic conditions such as osteoporosis.

A therapeutically effective amount of a senolytic agent can be from about 0.05 mg to about 1000 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, or any amount inbetween, of the senolytic agent. In certain embodiments, 50 mg of a senolytic agent is administered. In additional embodiments, the senolytic agent is Fisetin and is administered at about 1 μM to about 10004 in vitro. In still further embodiments, the senolytic agent is Fisetin and is administered to a human subject at about 1 mg/kg to about 100 mg/kg.

The amount of senolytic agent contained within the individual doses may be expressed in terms of milligrams of antibody per kilogram of patient body weight (i.e., mg/kg). For example, the senolytic agent may be administered to a patient at a dose of about 0.0001 to about 100 mg/kg of patient body weight.

Described herein is a three-pronged approach to the use of the elimination of senescent cells: i) eliminating senescent cells and stem cell banking and transplantation; ii) eliminating senescent cells in orthobiologic products; and iii) eliminating senescent cells in orthopedic surgery. Prong 1 is contemplated for outside of the body, prong 2 is contemplated for inside the body, and prong 3 is contemplated for both inside and outside of the body.

### Elimination of Senescent Cells and Stem Cell Banking and Transplantation

It is desirable to save stem cells for future use (referred to as “stem cell banking”), including to be given to a recipient (“transplanted”) for treatment of a condition. This banking often includes expansion. However, the regenerative potential of stem cells is reduced after ex vivo expansion (Monterras, et al. 2005 Science 309:2064-2067). It is hypothesized that when stem cells are banked, expanding them induces an aging effect in the cells. As a result, the banked and expanded cells injected into a subject typically have an increased proportion of senescent cells.

In one aspect, the disclosure provides methods for clearing senescent cells from banked stem cells in order to minimize the proliferation/accumulation of senescent cells upon (during) expansion of the banked cells. In additional embodiments, the banked cells are treated after expansion. In still further embodiments, the banked cells are treated between expansion cycles. In a preferred embodiment, the cells are treated before freezing down in liquid Nitrogen. By “clearing” senescent cells is meant targeting and killing them. “Clearing” does not necessarily mean that all senescent cells are cleared/killed. Rather, it means that the number of senescent cells in a subject after clearing is measurably lower than beforehand. In some embodiments, at least about 20%, at least about 30%, at least about 40%, at least about 50%, at least about 60%, at least about 70%, at least about 80%, at least about 90%, or at least about 95% of senescent cells are cleared. In additional embodiments, senotherapeutic agents may reduce senescence phenotypes in stem cells in the contexts described above not by clearing, but rather by rejuvenating health and function. Such senomorphic agents may, for example, suppress markers of senescence or the secretory phenotype of senescent cells without inducing cell death.

In one embodiment, senolytic agents are used to clear senescent cells from stem cells that have been banked. In a further embodiment, previously banked stem cells are treated with a senolytic agent in vitro. The treatment results in the killing of senescent cells. Senescent-free (or senescent-reduced) stem cells are purified and enriched for reintroduction into the subject from which the cells were originally harvested for banking. In one embodiment, thus purified and enriched senescent-free (or senescent-reduced) stem cells are reintroduced into a subject having eroded joint cartilage, for example, to treat hip arthritis.

Aging is associated with a depletion of functional stem cells, primarily due to cellular senescence and increased pro-inflammatory signaling. In order to be considered as a potential therapy for musculoskeletal repair, adipose-derived stem cells (ADSCs) require culture expansion before treatment. Similarly to aging, prolonged in vitro expansion of ADSCs results in a significant accumulation of senescent cells. In the instant embodiment, senolytic drugs may be used to attenuate senescence and produce an enriched stem cell population.

In additional embodiments, senescent cells can be quantified in bone marrow aspirate, bone marrow concentrate, and/or peripheral blood using flow cytometry. Late-stage senescent cells appear to be present in a greater quantity in the bone marrow aspirate than in the peripheral blood (data not shown).

### Elimination of Senescent Cells in Orthobiologic Products

The term “orthobiologics” or “orthobiologic products”, as used herein, refers to biologic substances that are used to improve healing of bone, cartilage, tendon, and/or ligament, for example, after injury or surgery. The products are deemed biologic, because they are made from substances naturally found in the body. Orthobiologics are advantageous in that they minimize the impact of degenerative disease and allow for more rapid recovery from musculoskeletal injury.

Orthobiologics typically include bone grafts, autologous blood, platelet-poor plasma (PPP), platelet-rich plasma (PRP), including derivatives with high or low leukocyte content (LR-PRP, LP-PRP), autologous conditioned serum, bone marrow aspirate concentrate (BMAC), and autologous stem cells. Orthobiologics may also include agents such as anti-fibrotic agents, senotherapeutics, fat grafts like microfragmented adipose tissue, nanofragmented adipose tissue, product derived from birth tissues, Extra cellular matrix (ECM) implants, supplements, alpha-2-macroglobulin (A2M), amniotic fluid, placental tissue, umbilical cord tissue, hyaluronic acid injections (or other viscosupplements), and stem cell injections.

In one embodiment, Fisetin treatment can be added to orthobiologics for patients with moderate osteoarthritis of the knee and/or hip, decreasing patient-reported pain and cartilage loss relative to patients that do not receive senolytic therapies with orthobiologics.

### Elimination of Senescent Cells in Orthopedic Surgery

Senescent cell burden has been shown to strongly correlate with age-related orthopaedic conditions. The injection of senescent cells is sufficient to drive age-related conditions such as osteoarthritis, frailty, and decreased survival. Thus, the development of therapies that selectively kill senescent cells is anticipated to delay the onset of aging phenotypes, attenuate severity of age-related diseases, improve resiliency, enhance survival, and extend lifespan (Xu, et al. 2018 Nat Med 24:1246-1256; Xu, et al. 2017 J Gerentol A Biol Sci Med Sci 72(6):780-785).

Furthermore, targeting and eliminating senescent cells has been shown to mitigate age-related musculoskeletal decline. Thus, in one aspect, the instant disclosure provides the treatment of patients with senolytic agents before stem cell harvesting in order to eliminate pre-existing senescent cells in the body. The subject is administered, for example, Fisetin or Quercetin. Fat or bone marrow or platelet-rich plasma cells are then harvested and expanded and/or enriched for re-injection into the subject. As a result, the surgical outcome of the subject is improved. For example, healing time is reduced, mobility is increased (for example, mobility of a joint is improved, as assessed by functional performance testing), cartilage of a joint is improved (as assessed by MRI, T2 mapping, or the like), pain is reduced (as assessed by PROs), scar tissue is reduced, and/or there is enhanced healing of soft tissue (i.e., following ACLR procedure), and/or senescence markers are reduced.

An improvement in a surgical outcome means a positive change from baseline. In this context, the term “positive” refers to a change associated with better healing or other clinical outcomes such as improved pain scores or mobility. As used herein, the term “baseline” means the numerical value of the parameter for a subject prior to or at the time of treatment according to the present invention.

To determine whether the surgical outcome/parameter has “improved,” the parameter is quantified at baseline and at one or more time-points after treatment. The difference between the value of the parameter at a particular time point following initiation of treatment and the value of the parameter at baseline is used to establish whether there has been an “improvement”.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, and pressure is at or near atmospheric.

### Example 1. Stem Cells Accumulate Senescence Upon Ex Vivo Culture Expansion

Serial passaging (expansion) increases cellular senescence in ADSCs. Senescence is detected in ex vivo-expanded cells. Because the re-injection of stem cells comprising senescent cells is undesirable, cells were evaluated for senescence after each passage. In order to determine the effect of culture expansion of adipose derived stem cells, passage 2 cells were thawed and cultured to passage 8. The cells were maintained in T-75 or T-175 culture flasks using normal growth media (DMEM:F-12 with 10% FBS and 1% penicillin/streptomycin). At 70-90% confluency, the cells were passaged and reseeded in the flasks at a 1:4 dilution. To test for senescence at passage 3, 4, 6, and 8, cells were plated in 12-well plates in triplicate at a density of 40,000 cells/well. After adhering overnight, the cells were treated with 100 nM of bafilomycin A1 for 1 hour at 37° C. and 5% CO₂. Next, 33 μM C12FDG was added and incubated for 2 hours. The cells were then washed and collected from the plate using the TrypLE reagent. Using the Guava EasyCyte flow cytometer, cells positive for the C12FDG substrate were quantified.

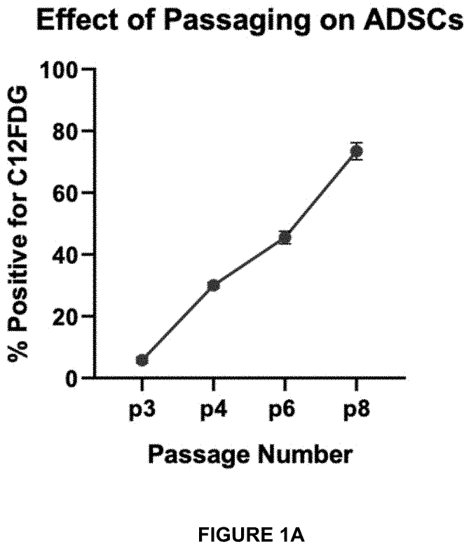

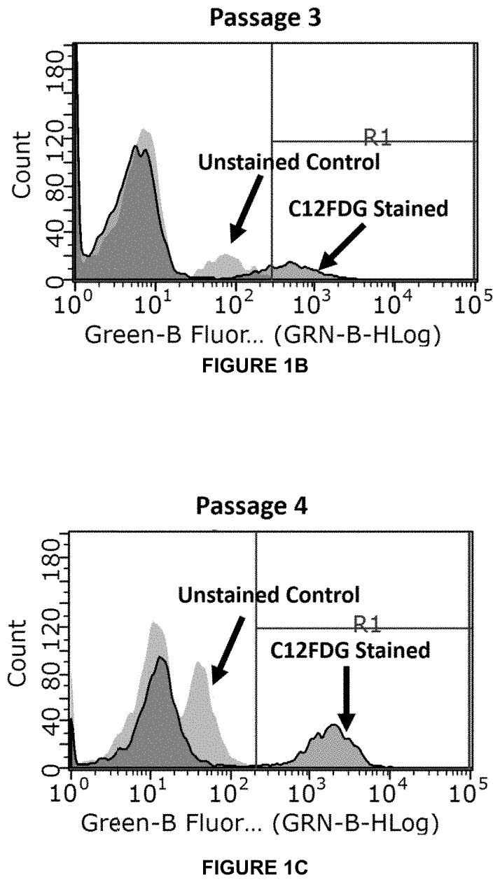

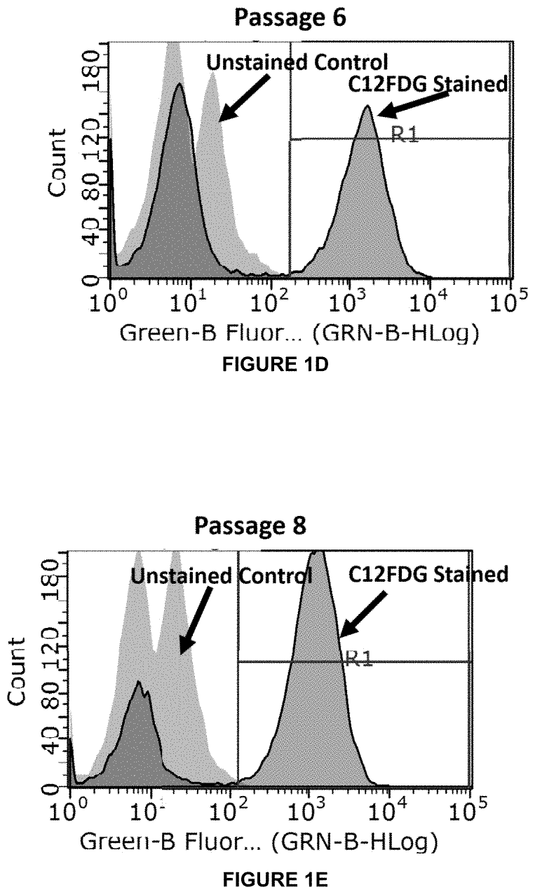

Senescence (% senescent cells) increased with each passage, with a spike from passage 3 to passage 4. FIG. 1A shows the effect of passaging on ADSCs. 6% of senescence at passage 3 (P3) spiked to 30% at passage 4 (P4) and further increased to 45% at passage 6 (P6). Thus, cells having been passaged more than four times were no longer appropriate for re-injection. Rather, banked cells cultured up to passage 3 are best for re-injection. Indeed, P1 to P4 are clinical grade cells. FIGS. 1B-E represent histogram plots for the C12FDG stain. R1 regions indicate cells positive for C12FDG.

Senescence associated secretory phenotype (SASP) concentrations increase with serial passaging. In order to determine the effect of passaging on the senescence associate secretory phenotype (SASP) and heterochromatin foci (SAHF), passage 2 cells were thawed and cultured to their respective passage numbers at testing. ADSCs were serially passaged 18 times and stained with antibodies for [symbol] -H2AX and H3K9. These antibodies bind senescence-associated heterochromatin foci (SAHFs), indicating gene silencing and DNA damage.

Specifically, the cells were maintained in T-75 culture flasks using normal growth media (DMEM:F-12 with 10% FBS and 1% penicillin/streptomycin). At 70-90% confluency, the cells were passaged and reseeded in the flasks at a 1:4 dilution. At their respective testing passage number, cells were plated into chamber slides for SAHF staining at a density of 5,000 cells/cm². Cells were seeded in triplicate for each group and incubated to adhere overnight. The following day, cells were fixed with cold 4% paraformaldehyde and stained with the H2AX antibody using a 1:150 dilution and the H3K9 antibody using a 1:250 dilution. DAPI was also used to stain the nuclei. Five images per well and three wells per group were imaged using the Nikon Eclipse Ni—U microscope. Cells positive for the H2AX and H3K9 antibodies were quantified using the ImageJ software.

For SASP detection, the media were collected after incubating with the cells for 24 hours, centrifuged at 2,000×g for ten minutes, transferred to new microcentrifuge tubes, and stored at −80° C. until testing. The Human Adipocyte Milliplex kit using the EMD Millipore Milliplex instrument was used to quantify the SASPs.

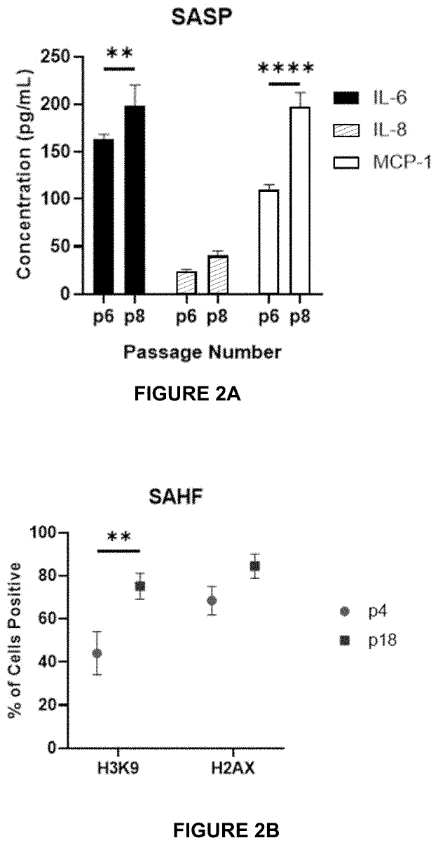

FIGS. 2A and 2B show that with increasing passage number, the senescence associated secretory phenotype (SASP) and heterochromatin foci (SAHF) also increase. IL-6, IL-8, and MCP-1 each increased in concentration from passage 6 to passage 8. Thus, it is shown herein that aging and culture expansion increase senescence levels. Indeed, aged individuals and culture-expanded cells had a higher number of senescent cells compared to young individuals.

### Example 2. Fisetin Treatment is Non-Toxic to Healthy Stem Cells

Fisetin is a natural flavonoid found in many fruits and vegetables. It is a known antioxidant and reducing agent due to its hydroxyl groups. It has been shown to reduce the secretion of several proinflammatory factors and has anti-cancer activity, blocking the mTOR and PI3K/AKT pathway, making Fisetin a strong therapeutic for targeting senescent cells. Fisetin has the molecular formula C15141006, molecular weight 286.24 g/mol, CAS name/number: Fisetin, 2-(3,4-dihydroxyphenyl)-3,7-dihydroxychromen-4-one, 528-48-3, and chemical structure:

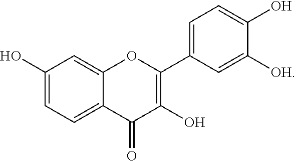 [SMILES: O=c1c(O)c(-c2ccc(O)c(O)c2)oc2cc(O)ccc12]

Since it would be deleterious to kill healthy, non-senescent cells when treating with Fisetin, it was necessary to determine the optimal Fisetin dose. In an effort to determine this concentration, healthy, low passage ADSCs were used to ensure no healthy cells were dying.

Reagents included ADSC culture media (CM) (DMEM-F12, Gibco #11320; 10% FBS, Gibco #16000044; 1% Penicillin Streptomycin, Gibco #15140122); 1×Ca and Mg Free DPBS (Gibco #14190250); TrypLE (Gibco #12605028); and Fisetin (Sigma #F4043).

### Adipose Derived Stem Cells

Banked ADSCs isolated from a 10-year-old male (YM), 27-year-old female (YF), 75-year-old male (OM), and 79-year-old female (OF) were purchased and cultured with normal growth media (DMEM/F:12, 10% FBS, and 1% penicillin/streptomycin).

### Fisetin Treatment

Fisetin was added to growth media and treated on cells for 24 hours. Following treatment, the media was changed to normal growth media for 24 hours before being used for cell toxicity, immunofluorescence, or flow cytometry.

### Immunofluorescence

Immunofluorescence results were generated to prove the concept of detecting senescence by nuclear staining. [symbol] -H2AX and H3K9 antibodies were used to stain senescence-associated heterochromatin foci. Each cell type was given 3 replicate wells, and each well was imaged 6 times, for a total of 18 images per group. Using an Image J program, the number of cells positive for each marker was quantified. The collected data was processed using one-way ANOVA and all but two treatment groups were found to be statistically relevant in every group besides [symbol] H2AX for passage 4 young female and passage 18 old female cells.

### Flow Cytometry

Cells were treated with 100 nM of bafilomycin A1 for 1 hour at 37° C. and 5% CO₂. Next, 33 μM C12FDG was added and incubated for 2 hours. Thus, cells were stained with the fluorescent senescence-associated marker C12FDG, collected, and tested for senescence using flow cytometry after 50 μM Fisetin treatment. 3 replicates were used per treatment. The InCyte software was used, and fluorescent gating was determined using unstained control cells.

Specifically, cells were thawed in a 37° C. water bath. In a biosafety cabinet, cells (1 mL) were collected and added to 9 mL of prewarmed ADSC culture media (CM, DMEM:F-12 with 10% FBS and 1% penicillin/streptomycin). The cells were centrifuged at 1000×g for 5 minutes. The media were aspirated, and the cells resuspended in 1 mL of CM. 104 of cell suspension was pipetted into a microcentrifuge tube. 104 of cell suspension were mixed with 104 of trypan blue. 10 uL of cell suspension/trypan blue were pipetted into a countess slide. The cell count was recorded using Countess II Automated Cell Counter. The volume required to seed 300,000 cells/T-175 flask was calculated. 20 ml of CM was added to a T-175 flask. The calculated cell suspension volume was added to the T-175 flask. The flask was placed in an incubator at 5% CO₂ and 37° C. The cells were allowed to adhere overnight. The flasks were brought back to the biosafety cabinet. The CM was aspirated, then washed by adding 10 mL of 1×DPBS. The DPBS was aspirated. 20 mL of prewarmed CM was added, followed by culture in incubator until 80% confluent, changing media every 2 days. At 70-90% confluent, the media were aspirated, followed by washing with 10 mL of 1×DPBS. 5 mL of TrypLE were added. The flask was placed in an incubator, and cell detachment was monitored with a microscope. Once detached, 5 mL of CM were added to the flask. The cell suspension was collected in a 15 mL conical tube and centrifuged at 1000×g for 5 minutes.

The media were aspirated, and the cells resuspended in 1 mL of CM. 104 of cell suspension was pipetted into a microcentrifuge tube. 104 of cell suspension were mixed with 104 of trypan blue. 10 uL of cell suspension/trypan blue were pipetted into a countess slide. The cell count was recorded using Countess II Automated Cell Counter. The volume required to seed 40,000 cells/well in a 12-well plate was calculated. 1 mL of CM was added per well, followed by the calculated cell suspension volume (to each well). The flask was placed in an incubator at 5% CO₂ and 37° C. The cells were allowed to adhere overnight. The flasks were brought back to the biosafety cabinet. The CM was aspirated, followed by washing by adding 1 mL of 1×DPBS per well. DPBS was aspirated. 1 mL of 50 uM Fisetin Supplemented Culture Media was added. The plate was cultured in an incubator for 24 hours. The plate was brought back to the biosafety cabinet. The media were aspirated followed by washing by adding 1 mL of 1×DPBS per well. DPBS was aspirated. 1 mL of prewarmed CM was added. The plates were incubated until cells are at 80% confluency. The next step was to proceed with downstream use.

Thus, inexpensive commercially available growth media products were used, cells were cultured in commercially available culture systems, single dose treatments reduced senescent cell populations, and culture expansion was possible without the accumulation of senescent cells.

### Cell Toxicity

The CellTiter 96® AQueous Cell Proliferation Assay was used to determine the toxicity of 50 μM, 25 μM, and 1 μM Fisetin on low passage (<p4) cells. Cells were seeded at 40,000 cells/well in a 12-well plate. After adhering overnight, the cells were treated with 50 μM, 25 μM, and 1 μM of Fisetin for 24 hours. After treatment, the media was changed back to normal growth media for another 24 hours. The cells were then treated with the CellTiter 96 AQueous Cell Proliferation Assay following the manufacturer's protocol. Values are relative to untreated, control cells.

### Results

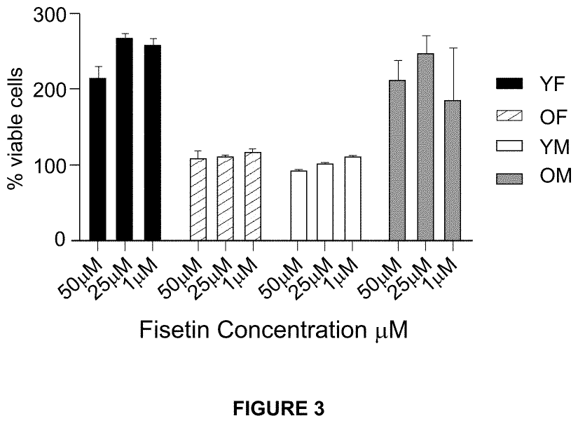

ADSC viability and proliferation was not hindered by Fisetin exposure. Fisetin was found to be non-toxic to ADSCs at 1-50 μM (FIG. 3). No significant difference in toxicity was noted among the treatment concentrations. 50 μM Fisetin treatment was not toxic to the health of low passage ADSCs.

### Example 3. Fisetin Treatment Results in Senescent Stem Cell Reduction

Immunosuppressors such as rapamycin have previously been shown to reduce cellular senescence while improving myogenic and chondrogenic differentiation in muscle-derived stem cells from progeroid mice. Thus, the senolytic agent and antioxidant Fisetin was studied for lowering senescence in ADSCs from aged, culture-expanded, and banked cells.

Adipose derived stem cells (ADSC), among other stem cells, can be promising tools for tissue regeneration and the prevention of age-related musculoskeletal degeneration. However, these age-related conditions also affect the ADSC populations in the form of cellular senescence. These senescent ADSCs, when isolated from senior individuals and/or culture-expanded and banked, can lead to a significant loss in therapeutic potential. Thus, there is reason to eliminate these cells during culture expansion, if a viable stem cell bank is to be generated for patients.

The use of senolytics has been shown to reduce the effects of age-related disease and improve lifespan. As a novel method for improving in vivo outcomes, senolytics prove promising; however, their use for the enrichment of culture-expanded and banked human stem cells has not been shown.

To determine Fisetin's senolytic ability in vitro, based on the data from the preceding example, the 50 μM Fisetin concentration was used to treat YF and OF cells. Senescent cells were quantified by immunofluorescence using [symbol] -H2AX and H3K9. Similarly, YM and OM cells were quantified using C₁₂FDG for flow cytometry. C₁₂FDG reacts with beta-galactosidase, a biomarker for senescence, to produce a fluorogenic substrate to be detected and quantified by flow cytometry.

### Methods

Cells were seeded in triplicate for each group using previously described culture methods and seeding densities. After adhering overnight, the media was changed. For the untreated group normal ADSC growth media was used (DMEM:F-12 with 10% FBS and 1% penicillin/streptomycin), and for the treated group 50 uM Fisetin was added. Cells were treated for 24-hours, after which the media was changed back to normal growth media for all groups. After culturing in normal growth media for another 24 hours, the media was collected for SASP quantification or the cells were processed for C12FDG or SAHF staining, as described in the preceding example.

Antibodies for H3K9 and H2AX (nuclear proteins involved in gene silencing of senescent cells) have been used to quantify the level of baseline senescence in stem cells from a 75-year-old male (“OM”). In order to determine the effect of Fisetin on removing senescent cells, ex vivo-cultured stem cells from the same OM were treated with 50 μM Fisetin. H3K9 and H2AX essentially represent senescent cells.

### Results

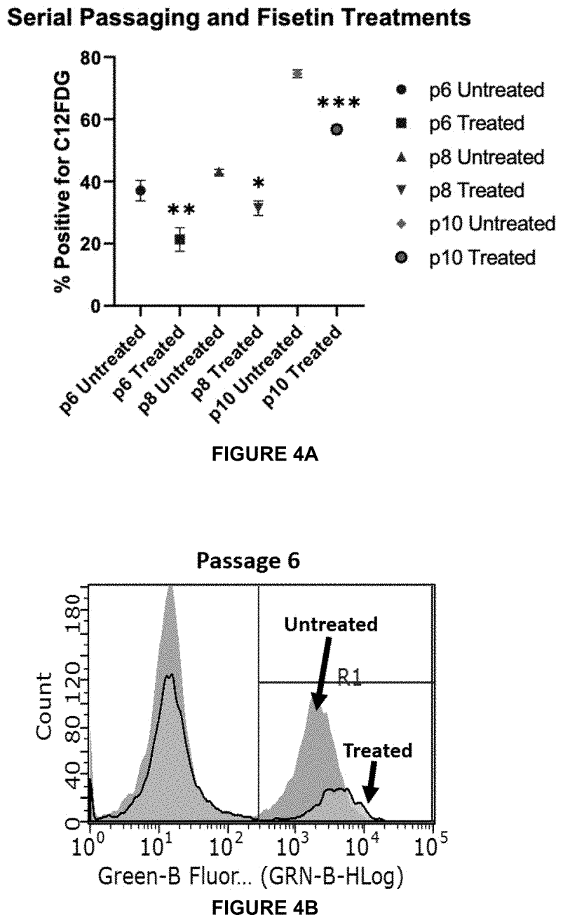

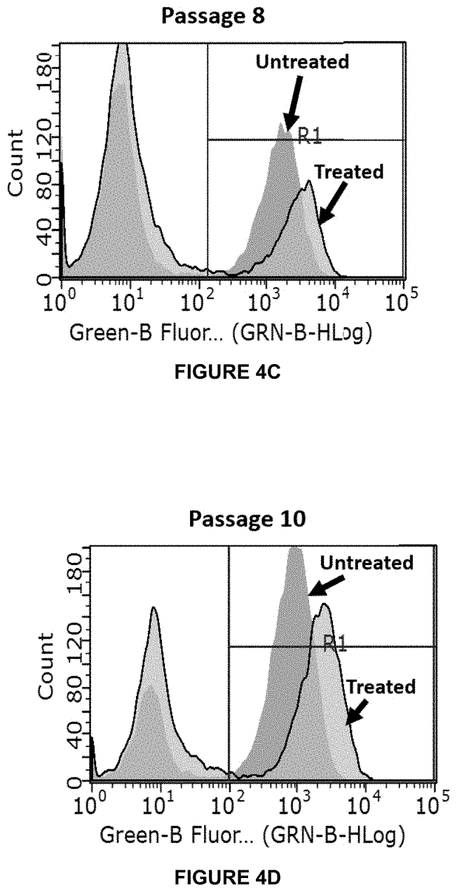

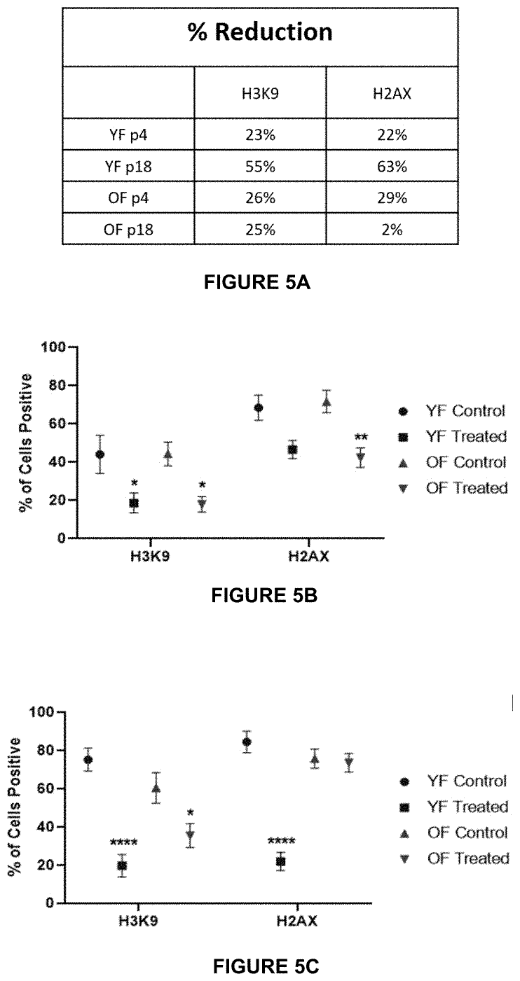

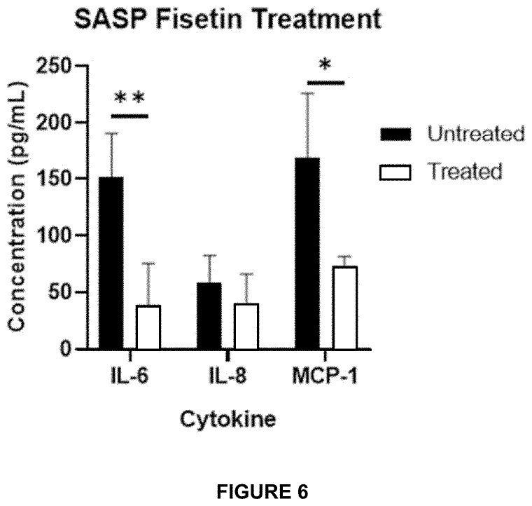

It is shown herein that Fisetin, a natural flavonoid and proven senolytic, could reduce senescence accumulation in culture-expanded and banked ADSCs by single and intermittent doses during culture expansion. This has been demonstrated herein by staining for senescence-associated β-galactosidase (C12FDG, FIGS. 4A-4D), senescence-associated heterochromatin foci (SAHF) H3K9 and [symbol] H2AX (FIGS. 5A-5C), and senescence-associated secretory phenotype (FIG. 6). These SAHFs are commonly found in senescent cells and indicate gene silencing and DNA damage, while β-galactosidase has been canonically used as a biomarker for cellular senescence due to its over-expression in senescent and aged cells.

Thus, Fisetin treatment reduced β-Galactosidase, as assessed via C12FDG staining (FIGS. 4A-4D). Within the R1 region of FIGS. 4B-4D, cells positive for C12FDG are noted by their respective peaks. The untreated cells are seen to present significantly larger peaks compared to their treated counterparts. The left half of each scan shows the untreated cells, with the “inside” peak representing senescent cells and showing a count of about 120. The right half of the scan shows the cells treated with 50 μM Fisetin, with the “inside” peak representing senescent cells and showing a count of about 20. Thus, senescence was significantly reduced in the Fisetin-treated cells. Furthermore, Fisetin treatment reduced H3K9 and [symbol] -H2AX Senescence Associated Heterochromatin Foci (SAHF) (FIGS. 5A-5C). The number of cells positive for H3K9 was significantly reduced in all groups (FIG. 5A). [symbol] -H2AX was reduced in low passage OF cells, low passage YF cells, and high passage YF cells. In all groups, a decrease was indicated. Finally, Fisetin treatment reduced the senescence associated secretory phenotype (SASP), as shown for cells treated with Fisetin, cultured with serum depleted culture media for 24 hours, of which the culture media was then assayed for SASPs using the Millipore Sigma Milliplex Instrument. 3 replicates were used for each treatment. (FIG. 6). Cytokines IL-6, IL-8, and MCP-1, known SASP factors, all showed a decrease in concentration after Fisetin treatment, with the decreases in IL-6 and MCP-1 especially significant.

### Example 4. Senescent Cells are Detected Using C₁₂FDG Staining and Flow Cytometry

The ability to accurately detect senescent cells and their associated Senescence-Associated Secretory Phenotype (SASP) factors could facilitate the understanding of individual patients' response to treatment and/or assist in deciding interventional strategies in the clinic. Senescence/senescent cell identification, quantitation, and/or characterization can be employed in each of the four above-identified prongs contemplated for elimination of senescent cells. In one aspect, senescent cells are detected and/or quantified using C₁₂FDG staining and flow cytometry.

The use of flow cytometry-assisted analysis of senescent peripheral blood mononuclear cells is known for clinical diagnostic use. Such analyses often rely on a specific subset of Peripheral Blood Mononuclear Cells (PBMCs), namely T-Cells, to reflect chronological age or senescence state in human whole blood. However, it is described herein, for the first time, that selecting all T-Cells (CD3+), which includes both CD4 and CD8 subsets from PBMCs, rather than one of those subsets, allows for the detection of reproducible and age-correlative changes in senescent cell number using C₁₂FDG staining.

C₁₂FDG (5-Dodecanoylaminofluorescein Di-β-D-Galactopyranoside) is the β-galactosidase substrate, which is covalently modified to include a 12-carbon lipophilic moiety. Once inside the cell by staining procedure, the substrate is cleaved by β-galactosidase enzyme, producing a fluorescent product that is well retained by the cells, likely by incorporation of the lipophilic tail within the cell membrane. C₁₂FDG has the molecular formula: C₄₄H₅₅NO₁₆, molecular weight: 853.9156, and the CAS name/number Dodecanamide, N-[3′,6′-bis(β-D-galactopyranosyloxy)-3-oxospiro[isobenzofuran-1(3H),9′-[9H]xanthen]-5-yl]-138777-25-0.

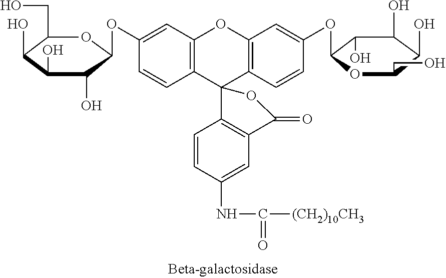

Senescence-associated beta-galactosidase (SA-β-gal or SABG) is a hypothetical hydrolase enzyme that catalyzes the hydrolysis of β-galactosidase into monosaccharides only in senescent and aging cells. Senescence-associated beta-galactosidase is a biomarker of cellular senescence due to its overexpression and accumulation, specifically in senescent and aging cells.

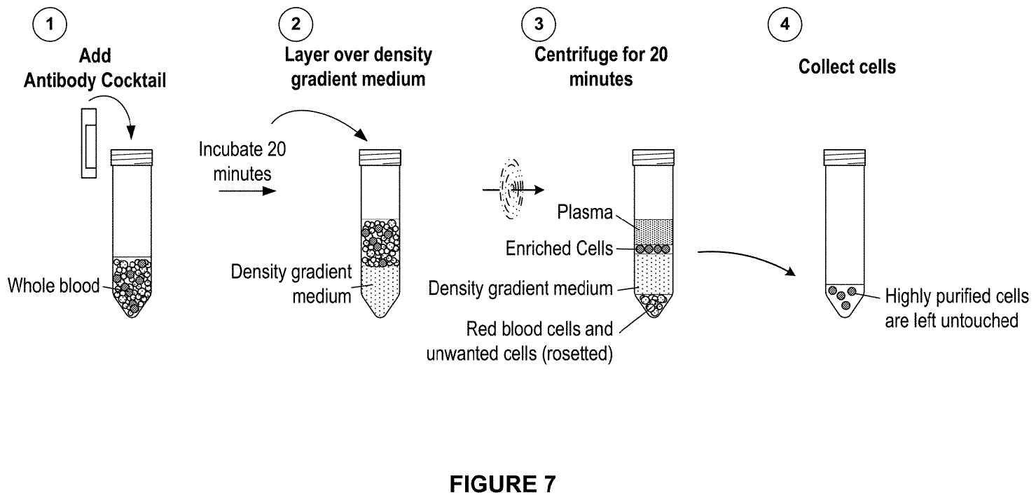

Whole blood was collected from a subject into 50 ml Syringe containing ACD-A and set on a rocker. SepMate tubes (Stem Cell Technologies) were prepared with 15 ml Lymphoprep in a sterile environment. 10 ml whole blood were aliquoted into 50 ml conical tubes. 500 μl (50 ul/ml) RosetteSep (Stem Cell Technologies) was added to 10 mL sample of blood. For other blood samples, total PBMCs were collected as described below without T-cell enrichment for comparison. The mixture was incubated 10 min at room temperature (RT). An equal volume dilution medium (DM—sterile PBS and 2% FBS) was added to the sample and mixed gently. The diluted sample was slowly added to the SepMate tube containing Lymphoprep density gradient medium. The mixture was centrifuged at 1200×g for 10 minutes, brake on. The enriched cells were collected by pouring supernatant into a new tube. The enriched cells were washed by topping off with DM and then centrifuged at 300×g for 10 minutes with brake low (set at 4). The supernatant was discarded, and the cells were gently reconstituted in 10 ml DM. The last two steps were repeated. The T-cell enrichment protocol is schematically depicted in FIG. 7. The pellet was reconstituted in 4 mL C12 Culture Media and distributed evenly into two 15 ml conical tubes. One was treated with 30 μM C12FDG, the other remained the unlabeled control. The caps were loosened, and the tubes were incubated for 1 hr, followed by invert mix 1× at 30 min in a tissue culture incubator with 5% CO₂. The cells were spun and washed 2× with 5 ml cold PBS (300×g with full brake). They were then reconstituted in 1 mL PBS on ice protected from light.

The Guava EasyCyte HT was utilized to analyze C12FDG cells via flow cytometry. The Guava loading was according to the following well assignment scheme example (Table 1, below):

TABLE 1

| Well | Study ID | Treatment |
|---|---|---|
| A1 | P001 | Unlabeled |
| A2 | P001 | C12 Labeled |
| A3 | P001 | C12 Labeled |
| A4 | P001 | C12 Labeled |
| A5 | N/A | DI Water |
| B1 | P002 | Unlabeled |
| B2 | P002 | C12 Labeled |
| B3 | P002 | C12 Labeled |
| B4 | P002 | C12 Labeled |
| B5 | N/A | DI Water |

Upon setting the threshold and gain controls, samples were acquired in triplicate. The run was saved as an FCS file, and the gate to exclude the labeled control. Anything within the gate was positive for C₁₂-FDG.

### Analysis

In order to assess the percent, number, and concentration (cell/ml) of senescent CD3⁺ T-cells, the setting was adjusted using an unstained control (i.e., cells not treated with C₁₂FDG). First, thresholds were set for FSC and SSC to catch cellular events. Next, the green channel gain controls were set so that the background events fell between the 1st and second decade. Finally, it was ensured that PBS alone (vehicle) would not register events with the adjusted setting parameters.

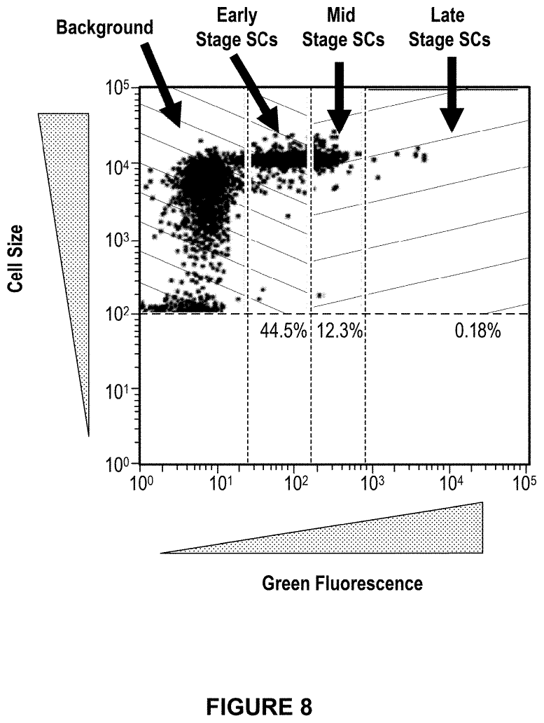

Samples were run in triplicate wells with water-only runs every 5 wells. Typically, distinct “subpopulations” of senescent cells having varying degrees of intensity were found (FIG. 8). These represent very “bright” cells that exhibit intense fluorescent signal due to high enzymatic breakdown of the C₁₂FDG molecule. These cells are thought to be “late stage” senescent cells, which may be the most disruptive cells physiologically. In a further embodiment, a cell sorter could be used to pick (sort/accumulate) cell subsets categorized as early stage senescent cells, mid-stage senescent cells, and late stage senescent cells. In a therapeutic setting (pre-clinically or clinically), sorted cells may be used to optimize the collection of healthy cells or non-senescent cells prior to autologous or allogenic transplantation.

The advantages to the rapid assay include i) the use of C₁₂FDG as a fluorescent stain (as opposed antibody staining immunophenotyping); ii) the use of benchtop flow cytometry; iii) the complete assay could be run more quickly (in a matter of hours) due to the dynamics of the stain; iv) the sample required is a small volume (as little as 2-5 ml blood); v) use of CD3⁺ T-Cell populations or total PBMC populations; vi) ability to detect changes following senolytic treatment; vii) ability to correlate senescent cell number with certain inflammatory factors and/or SASPs in plasma; viii) use with BMA and whole blood; and ix) sensitivity to potential stages of senescence.

### Example 5. Fisetin Treatment Leads to Reduction in Senescent Cells in an 82-Year-Old Male

Senescent cells were treated with Fisetin and then further characterized using flow cytometry (as described in Example 4, above). Peripheral Blood CD3⁺ T-cells from an 82-year-old male before & after Fisetin treatment (100 mg per day for 45 days) were evaluated for senescence and for senescent cell populations. Before treatment, the subject measured about 29% senescent cells, and after treatment, the subject measured about 4% senescent cells (data not shown). Thus, the number of senescent cells was significantly decreased by treatment with the senolytic agent Fisetin, as shown in Table 2, below.

TABLE 2

| CDR+ cells at baseline | CD3+ cells after Fisetin treatment |
|---|---|
| % senescent cells = | % senescent cells = |
| 28.89% +/− 1.09% | 3.82% +/− 1.46% |
| Total senescent cells = | Total senescent cells = |
| 14445 +/− 55 | 191 +/− 73 |
| Senescent cell concentration = | Senescent cell concentration = |
| 25804 +/− 2841 cells/mL | 7397 +/− 3191 cells/mL |

### Example 6. Further Characterization of Senescent Cells (“Senescence Profiling”)

### Venipuncture (Blood Draw)

A certified phlebotomist used a 19-gauge butterfly needle to perform a standard venipuncture from the antecubital (AC) area, forearm or hand vein. Approximately 35 mL of blood was drawn into one 30 cc syringe (with ACD-A) and one 5 cc (without ACD-A) syringe. Blood was used for CBC count (not shown), multiplex immunoassays (data not shown), T-Cell Expression Assay (data not shown), and C12FDG FLOW assay using enriched T-Cells and total PBMCs. For total PBMCs and enriched T-Cells, SepMate™ tubes (Stem Cell Technologies) were used following manufactures protocol with Lymphoprep™ (Stem Cell Technologies) gradient centrifugation. Specifically, in separate tubes, CD3⁺ T-Cells were enriched using the RosetteSep™ commercial kit following manufactures instructions (Stem Cell Technologies #15021). A portion of cells (enriched T-cells and total PBMCs) were stained with 30 μM C12FDG in general culture media supplemented with 10% FBS and analyzed with flow cytometry (Guava EasyCyte, Luminex). To assess the percent, number, and concentration (cell/ml) of senescent CD3+ T-cells and PBMCs, settings were adjusted first using an unstained control (i.e., cells not treated with C12FDG). First, thresholds were set for FSC and SSC to catch cellular events. Next the green channel gain controls were set so that the background events fall between the 1st and second decade. Finally, it was ensured that PBS alone (vehicle) did not register events with the adjusted setting parameters. Samples were ran in triplicate with water-only runs every 5 wells.

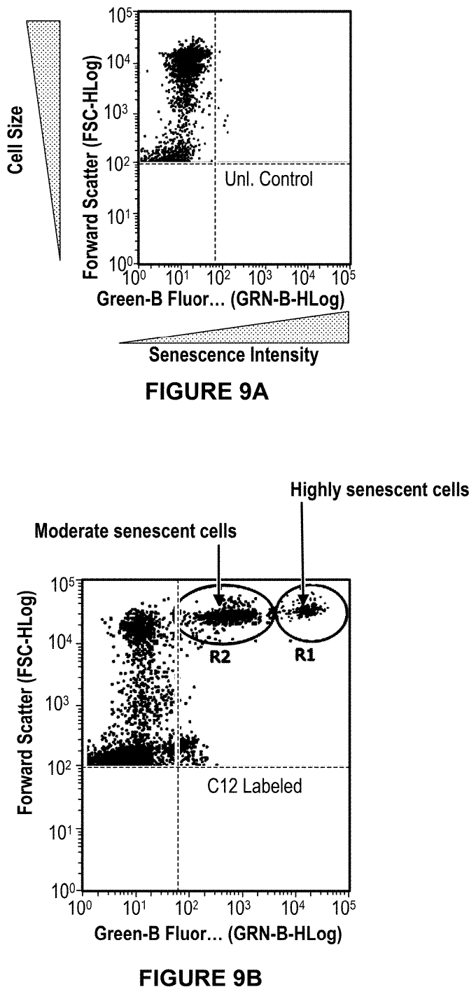

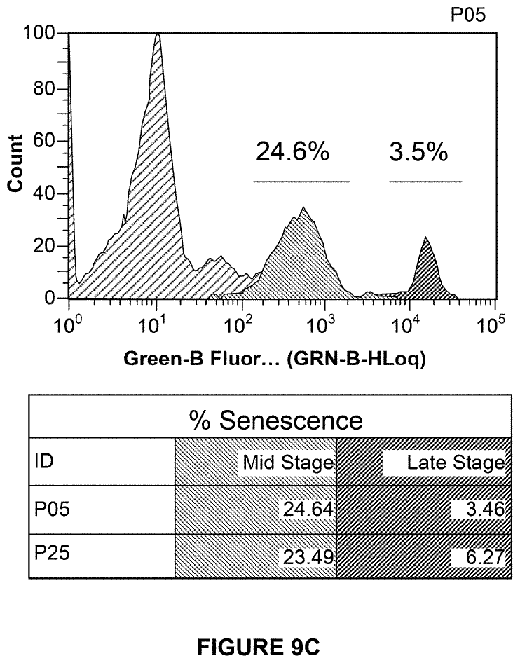

FIG. 9A shows control, unlabeled cells. Without the labeling, the senescent cells are not detected. The staining of T cells revealed distinct populations of high intensity, bright cells and lower intensity dim cells. This difference in intensity is due to the fluorescent marker C12FDG only fluorescing when hydrolyzed by an enzyme upregulated during senescence—β-galactosidase—leading to a range of signal because of the range of expression of the latter. The populations represent stages of senescence, in which the brightest events are late-stage senescent (high C12FDG signal) and possibly more pathological.

In one embodiment, senescent cells can be further characterized. In another embodiment, senescent cells can be characterized before and after treatment with a senolytic agent. FIGS. 9B and 9C show that the senescent cells are made up of populations of moderate senescent cells and highly senescent cells, wherein the moderate senescent cells correspond to mid-stage senescence, and the highly senescent cells correspond to late-stage senescence.

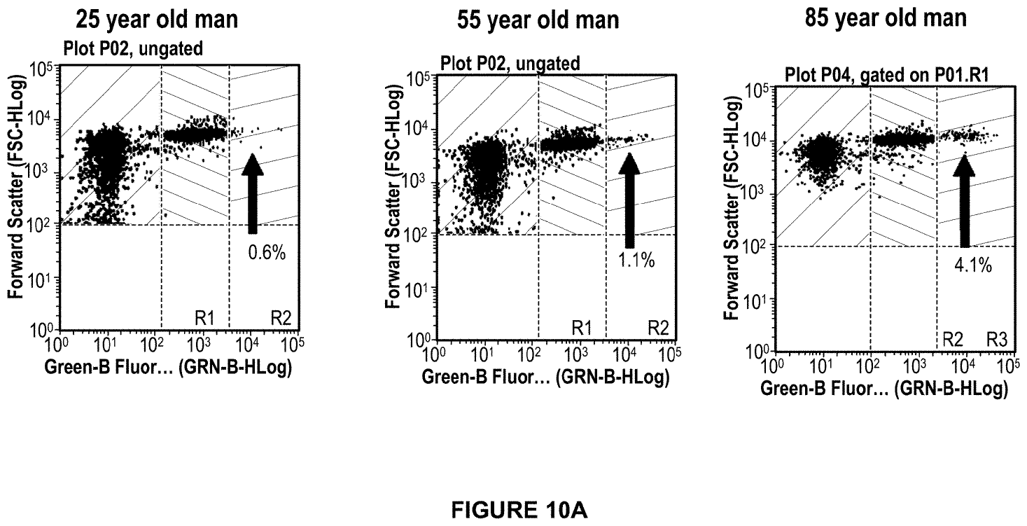

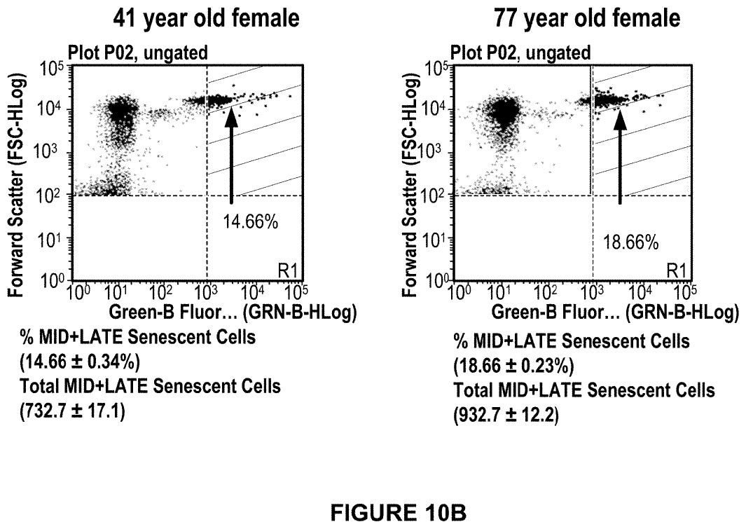

The senescent cell make-up (e.g., mid- and late-stage senescent cells) was compared between a 25-year-old male, a 55-year-old male, and an 85-year-old male. The samples evaluated were peripheral blood samples. FIG. 10A shows that the 25-year-old male had a lower percentage of late-stage senescent cells (0.6%) than the 55-year-old male, who had a lower percentage of late-stage senescent cells (1.1%) than the 85-year-old male (4.1%). FIG. 10B show that a 41-year-old female had a lower total number of mid+late stage senescent cells, a lower total number of late-stage senescent cells, a lower percentage of mid+late stage senescent cells, and a lower percentage of late-stage senescent cells, vs. a 77-year-old female.

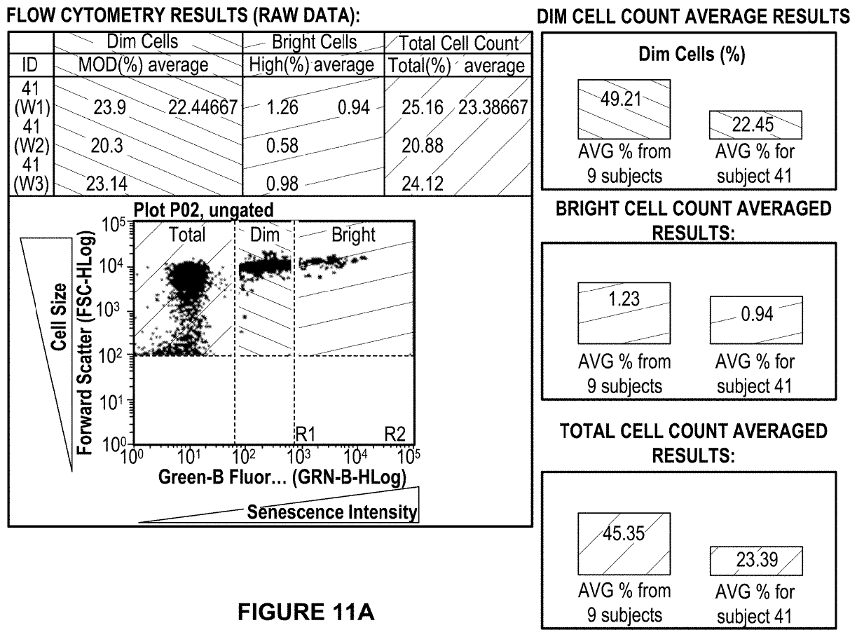

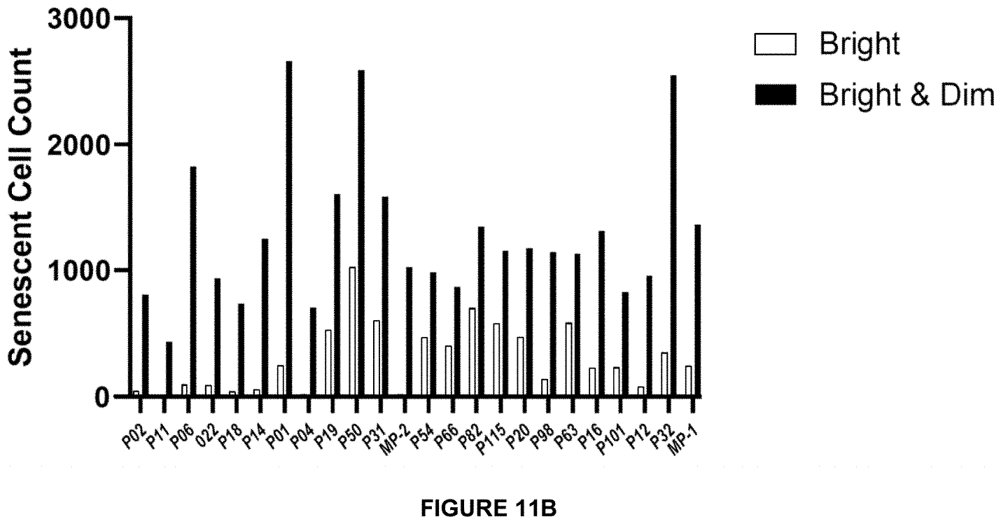

Senescence profiling was carried out for 67 subjects, including male and female subjects of different age groups. Peripheral blood was collected from each subject and T-cells were enriched in each sample for evaluation of senescent cells. Total, dim, and bright cells (senescent cells) were measured by using C12FDG cellular stain and analysis on flow cytometer equipment. Raw data obtained from scatter plots were gated into two areas of interest: i) dim cells (events) and ii) bright cells (events). As described below in “Example of Interpretation of Results”, each subject received their senescence profiling results in a report in the format described earlier. The percentage of bright senescent cells versus the percentage of dim senescent cells was evaluated. Each subject received their senescence profiling results in a report in the format of FIG. 11A. For subjects with multiple blood draws (longitudinal study), the report would reflect changes over time in senescent cells.

Analysis of stained blood cells (T-Cells) revealed distinct sub-populations of high intensity or “bright” cells and lower “dim” cells. The fluorescent marker of interest (C12FDG, senescence marker compound) only fluoresces when hydrolyzed by an enzyme upregulated during senescence (β-galactosidase), leading to a range of signal due to ranges of β-galactosidase expression. This suggested that these populations represent stages of senescence, whereby the brightest events are the more late-stage senescent (high C12FDG signal) and, thus, potentially more pathological. In one embodiment, these late-stage or “bright” cells are indicative of health status, specifically, poorer health or risk for greater risk for age-related and/or senescence-related diseases/disorders. This includes musculoskeletal conditions such as (but not limited to) osteoarthritis, osteoporosis, osteopenia, frailty risk, sarcopenia, tendinopathies, arthritis, muscle strength loss. This also includes other age-related co-morbidities such as type 2 diabetes, metabolic syndrome, neurodegenerative diseases including cognitive decline, and heart disease including atherosclerosis. The data of the group of subjects is summarized in FIG. 11B. The percentage of bright senescent cells was significantly lower than the percentage of dim senescent cells (save a few outliers being further investigated).

### Example 7. Clearing Senescent Cells Using Senolytic Agents Delayed Osteoarthritis in Progeria Mice

Hutchinson-Gilford progeria syndrome (HGPS) is an autosomal dominant disease that involves premature aging (lamin A deficiency), causing early death in childhood. Rapid sclerotic skin, joint contractures, bone abnormalities, and growth impairment are observed in subjects having the disorder.

Zmpste24−/− (Z24−/−) mice constitute a reliable animal model of HGPS. They are incapable of producing lamin A, an essential component of the nuclear lamina. Premature senescence was observed in the Zmpste24−/− mouse model—indeed, an accumulation of senescent cells was observed in the mouse.

The effect of senolytic drugs on osteoarthritis in Zmpste24−/− mice was investigated.

### Senolytic Drug Treatments In Vivo

Adult progeroid Z24−/− mice were administered senolytic drugs via oral gavage (150 μl volume) starting at 2.5 to 3.0 months of age. D/Q (5 mg/kg; 50 mg/kg in 10% PEG400/5.0% DMSO) was given as a single dose at 2.5 months or multi-dose at 2.5, 3.0, 3.5, 4.0, 4.5, and 5.0 months. Both single and multi-dose DQ mice were sacrificed at 5 months. FIS (100 mg/kg in 90% PEG400/10.0% ethanol) treated mice were given a total of 4 weekly doses of drug starting at 3.0 months of age, then sacrificed at 4.0 months of age. For 17-DMAG treatments (10 mg/kg, 1% DMSO-PBS), mice were treated 3 times per week starting at 2 months of age then sacrificed at 4 months of age.

### Histological Evaluation of Cartilage

Knee joints from Z24−/− animals were decalcified using 10% ethylenediaminetetraacetic acid disodium (EDTA) plus 1% sodium hydroxide for 6 weeks. Whole knee joints were cut, dehydrated, and then paraffin-embedded, so that the middle of the joint (groove level) and the edge of the joint (condyle level) could be viewed sagittally. To interrogate articular cartilage, paraffin sections of 5 μm were cut and stained with alcian blue (to detect acidic glycosaminoglycans GAGs; mucopolysaccharides) and safranin-O (to detect proteoglycan content). Images were captured for entire area of cartilage surface of each animal at both middle (groove) and edge (condyle) at ×200 magnification. Images were obtained using a Leica DMIRB microscope equipped with a Retiga digital camera and evaluated using Northern Eclipse software (v6.0; Empix Imaging).

### Immunological Staining of Cartilage and Primary Chondrocytes

Immunohistochemistry staining of p16INK4a positive cells was used to evaluate senescent chondrocyte number in 5 μm paraffin sections from the knee joint. Briefly, after deparaffinization, washing, and blocking with 5% donkey serum in PBS, sections were incubated with rabbit anti-p16 antibody (Abcam, 1:100) in 5% donkey serum overnight. Subsequently, immunoreactivity was detected using goat anti-rabbit biotin (BA 1000, Vector Laboratories, Burlingame, Calif., USA, 1:200 dilution), incubation with ABC reagent (PK 7200, Elite ABC kits, Vector Laboratories), then visualized via diaminobenzidine (DAB) staining (SK-4100, Vector Laboratories) following manufactures instructions.

To detect abnormal cell nuclei in primary chondrocytes, immunofluorescence was performed. Cultured cells were fixed in 4% paraformaldehyde and permeabilized with 0.2% Triton X-100 (Sigma-Aldrich, St. Louis, Mo., USA). The cells were then incubated with Block Ace Solution (Dako Japan, Kyoto, Japan) and stained with antibodies against lamin A/C (1:50; Santa Cruz Biotechnology) as a sensitive marker for the nuclear lamina, followed by incubation for 1 h with an Alexa Fluor 594-conjugated rabbit anti-goat IgG1 (1:1,000; Molecular Probes, Grand Island, N.Y., USA). The nuclei were counterstained with DAPI solution (1:500; Sigma-Aldrich) for 5 min, in order to visualize the nuclei. Images were obtained with a Leica DM IRB microscope equipped with a Retiga digital camera and were evaluated with Northern Eclipse software (v6.0; Empix Imaging).

### Micro-CT Analysis

For Fisetin treatments, mice were imaged using the eXplore Locus Utra Pre-Clinical CT Scanner (GE Healthcare, London, ON) through the pre-Clinical CT Core Facility with the following acquisition settings: 140 kVp, 22 mA with 16 s rotation/exposure. Non-cardiac gated CT images were acquired at all-time points. Simple back projections were obtained from the 0.154 μm image reconstruction and exported as DICOM images. Image analysis was performed using the OsiriX software. Qualitative analysis of the bones was performed in 2D and 3D reconstructions using OsiriX. MicroView imaging software was also used for the quantitative analysis of the bone mineral density.

While no significant protective effects in bone were observed using D+Q, the use of the phytonutrient Fisetin was effective. Z24−/− mice were treated with Fisetin weekly for 4 weeks (100 mg/kg) starting at 3 months of age then sacrificed at 4 months of age. 4 months was chosen as a sacrifice time (versus 5 months) given the more advanced pathology that occurred at 5 months, a time when therapies might have negligible effects like seen in D+Q treated animals. Z24−/− mice treated with FIS were found to have reduced transcript levels of the senescence marker p16INK4a and SASP factors IL-6 and TGF-β (data not shown). In addition to reduced senescence markers in tissues, weekly dosing of FIS for weeks starting at 3 months of age were able to significantly attenuate bone density loss in Z24−/− mice as evidenced by micro-CT analysis. Significant higher scores were observed for Hounsfield unit (HU) intensity, bone mineral density (BMD), and specific bone surface (BS/BV) in Z24−/− mice treated with Fisetin versus untreated Z24−/− mice. Thus, unlike D+Q, senolytic therapy with Fisetin protects against age-related bone density loss suggesting the importance of senescence to bone loss during aging.

### Example 9. Bone Marrow Aspirate Concentrate with Senotherapeutic Flavonoid to Improve Articular Cartilage Repair

Microfracture (MFx) is the most commonly used first-line treatment for cartilage injuries and has been shown to have inferior long-term clinical outcomes, primarily due to the production of fibrocartilage repair tissue. Losartan administration was found to enhance microfracture for cartilage repair (Utsunomiya, et al. 2020 AJSM 48(4):974-984). Bone marrow aspirate concentrate (BMAC) is another biological product that has demonstrated positive effects in bone and cartilage applications, including osteochondral defect, osteoarthritis and bone nonunion. Furthermore, early clinical data suggests BMAC may help stimulate a more robust hyaline cartilage repair tissue response through both chondrocyte differentiation of MSCs and paracrine signaling. Previous studies have reported that Fisetin decreased cartilage destruction and subchondral bone plate thickness and relieved synovitis in mice OA models. However, the synergistic effect of adding BMAC and losartan to Fisetin has not yet been investigated in osteochondral models. The purpose of this example was to determine the effect of Fisetin, as well as its synergistic effects with losartan and BMAC, for cartilage repair in a rabbit model.

### Methods

BMAC Collection and Processing for Autologous Transplantation: Bone marrow aspirate (BMA) was collected through iliac crest aspiration in each rabbit and processed using a benchtop centrifuge to prepare BMAC.

Surgical procedure: 48 skeletally mature New Zealand White Rabbits were anesthetized to create a 5 mm diameter full thickness osteochondral defect (OD) in the patella groove (depth: 2 mm). Microfracture procedures were performed within the defects by creating 5 holes spaced equally apart and 2 mm deep into subchondral bone with a micro-awl. After microfracture surgery, 0.5 ml of BMAC was injected intra-articularly in the left knee. 6- and 12-week studies were performed comparing the eight conditions listed below, using 6 rabbits in each group. At each end point, rabbits were euthanized for micro CT, qPCR and histological analysis.

### Results

Preliminary data showed that the augmentation of BMAC, or Fisetin and Losartan, to microfracture accelerated cartilage regeneration in a rabbit osteochondral defect model (data not shown). Thus, using senotherapeutic agents like Fisetin, senescent cells can be eliminated from the orthobiologic BMAC, and this enrichment can potentially revitalize stem cells in BMAC and improve their regenerative potential.

### Example 10. Fisetin Reduces Early- and Late-Stage Senescent Cells

Five patients with an age range from 56-77 years were treated with Fisetin at 100 mg/day for 30-45 days. Of note, patients with “high” baseline numbers of C12FDG+ T-Cells, or “bright” cells, showed a 10-20 fold reduction in “bright” C12FDG+ T-cells (data not shown). At passage 19 (P19), pre-treated/untreated bright cells measured a mean of 12.53%, while treated/post-treated bright cells measured a mean of 1.15%. At passage 50 (P50), pre-treated/untreated bright cells measured a mean of 19.80%, while treated/post-treated bright cells measured a mean of 0.60%.

In the moderately senescent, or “early” stage senescent cell populations (i.e., those staining more dim for C12FDG), there was also a 1-2 fold reduction in the percent of C12FDG+ cells (data not shown). At passage 19 (P19), pre-treated/untreated dim cells measured a mean of 54.22%, while treated/post-treated dim cells measured a mean of 26.70%. At passage 50 (P50), pre-treated/untreated dim cells measured a mean of 59.59%, while treated/post-treated dim cells measured a mean of 27.55%.

Overall the data demonstrated 1) the sensitivity of the methodology described herein to detect changes in senescent cells using known senescent cell targeting drugs (senolytics), and 2) the ability of orally administered Fisetin to significantly reduce the number of “bright” or “highly senescent” cells, cells which are thought to be primary drivers of senescence-associated pathology during aging. In some embodiments, patients with high baseline bright/late-stage senescent cells respond especially significantly to senotherapeutic treatment.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of detecting senescent cells in a cell sample, the method comprising staining the cells with C12FDG; and subjecting the cells to flow cytometry.

2. The method of claim 1, wherein the cell sample comprises CD3+ T cells.

3. The method of claim 1, wherein the cell sample is total peripheral blood mononuclear cells, bone marrow aspirate (BMA), whole blood, ex vivo culture-expanded stem cells, banked stem cells, freshly isolated stem cells, or endogenous stem cells.

4. The method of claim 1, wherein the detected senescent cells are characterized according to stage of senescence.

5. The method of claim 4, wherein the characterization is based on brightness of signal.

6. The method of claim 4, wherein the stage of senescence is early-stage (relatively low C12FDG positivity), mid-stage (relatively moderate C12FDG positivity), or late-stage (relatively high C12FDG positivity), as determined by normalized event gating with flow cytometry.

7. A method of removing senescent cells from banked stem cells, the method comprising adding at least one senolytic agent to the cells.

8. A method of enriching banked stem cells, the method comprising adding at least one senolytic agent to the cells.

9. The method of claim 7, wherein the banked stem cells are selected from the group consisting of ADSCs, ex vivo culture-expanded stem cells, freshly isolated stem cells, endogenous stem cells, bone marrow aspirate concentrate (BMAC) stem cells, and whole blood stem cells.

10. The method of claim 7, wherein at least 25% of the senescent cells are removed.

11. The method of claim 7, wherein at least 50% of the senescent cells are removed.

12. The method of claim 7, wherein at least 75% of the senescent cells are removed.

13. The method of claim 7, wherein at least 80% of the senescent cells are removed.

14. The method of claim 7, wherein at least 85% of the senescent cells are removed.

15. The method of claim 7, wherein at least 90% of the senescent cells are removed.

16. The method of claim 7, wherein at least 95% of the senescent cells are removed.

17. A method of treating a disease, disorder, or condition associated with senescence in a subject, the method comprising administering at least one senolytic agent to the subject.

18. A method of treating a disease, disorder, or condition associated with senescence in a subject, the method comprising removing stem cells from the subject, culturing the cells with at least one senolytic agent, and administering the cultured cells to the subject.

19. A method of treating an age-related disease, disorder, or condition in a subject, the method comprising removing stem cells from the subject, culturing the cells with at least one senolytic agent, and administering the cultured cells to the subject.

20. The method of claim 17, wherein the disease, disorder, or condition is osteoarthritis.

21. A method of improving a surgical outcome in a subject, the method comprising administering at least one senolytic agent to the subject.

22. The method of claim 21, wherein the senolytic agent is administered to the surgical site in the subject.

23. A method of improving a surgical outcome in a subject, the method comprising removing stem cells from the subject, culturing the cells with at least one senolytic agent, and administering the cultured cells to the subject.

24. The method of claim 23, wherein the cultured cells are administered to the surgical site in the patient.

25. The method of claim 21, wherein the surgical outcome is wound healing.

26. The method of claim 22, wherein the surgical site is a wound.

27. A method of removing senescent cells from an orthobiologic product, the method comprising adding at least one senolytic agent to the product.

28. The method of claim 7, wherein the at least one senolytic agent is Fisetin.

29. A pharmaceutical composition comprising at least one senolytic agent for use in the treatment of an age-related disease, disorder, or condition.

30. A pharmaceutical composition comprising at least one senolytic agent for use in the treatment of a disease, disorder, or condition associated with senescence.

31. A pharmaceutical composition comprising at least one senolytic agent for use in the improvement of a surgical outcome.

32. The composition of claim 29, wherein the at least one senolytic agent is Fisetin.

33. A method of treating a subject having a senescence-related condition or disease, the method comprising measuring senescence in the subject or in a tissue sample from the subject and administering a senolytic agent to the subject.

34. The method of claim 33, wherein the subject or the tissue sample of the subject comprises at least about 4% senescent cells.

35. The method of claim 33, wherein the senolytic agent is Fisetin.
